(19) United States
(12) Patent Application Publication
(10) Pub. No.: US 2026/0366059 A1
(43) Pub. Date: Nov. 19, 2026

(54) METHOD FOR IDENTIFYING A HYPEROVULATORY DONOR EWE AND USE THEREOF

(71) Applicant: CHINA AGRICULTURAL UNIVERSITY, Beijing (CN)

(72) Inventors: Guangyin XI, Beijing City (CN); Jianhui TIAN, Beijing City (CN); Qin Li, Beijing City (CN); Zhengrong WAN, Beijing City (CN); Lei AN, Beijing City (CN); Rong LI, Beijing City (CN); Min GUO, Beijing City (CN)

(21) Appl. No.: 19/722,418

(22) Filed: Jun. 26, 2026

Related U.S. Application Data
(63) Continuation of application No. PCT/CN2026/076106, filed on Jan. 30, 2026.

(30) Foreign Application Priority Data
May 7, 2025 (CN) 202510584403.3

(51) Int. Cl. C12Q 1/37; A01K 67/02; G01N 33/49

(52) U.S. Cl. CPC C12Q 1/37; A01K 67/02; G01N 33/491; A01K 2227/102; G01N 2333/916; G01N 2333/948

(57) ABSTRACT

The present disclosure relates to a method for identifying a hyperovulatory donor ewe and use thereof. It provides a standardized identifying approach that avoids insufficient ovulation and large inter-donor variations caused by subjective judgment in conventional methods. The method enables efficient donor ewe selection, improves embryo production, reduces labor and material inputs, significantly reduces breeding costs, and enhances economic benefits. It is highly important for promoting industrial application of livestock embryo production technology. Moreover, the method has the advantages of simple operation, high accuracy, high efficiency, convenience for popularization.

### CROSS-REFERENCE TO RELATED APPLICATION

The present application claims priority to Patent Application No. CN202510584403.3 filed on May 7, 2025, entitled “METHOD FOR IDENTIFYING A HYPEROVULATORY DONOR EWE AND USE THEREOF”, which is incorporated herein by reference in its entirety.

### TECHNICAL FIELD

The present disclosure relates to the field of livestock breeding technology, and particularly, to a method for identifying a hyperovulatory donor ewe and use thereof.

### BACKGROUND

Embryo engineering technology refers to modern biological techniques that perform a variety of microscopic manipulations and treatments on animal early embryos or gametes (sperms or oocyte). It includes in vitro fertilization, embryo transfer, embryo segmentation, embryo cryopreservation, sex control, nuclear transfer, and the like. Through techniques such as embryo production and transfer, the reproductive potential of the elite ewe breed can be fully utilized to increase the annual progeny output by 30-40 times, thereby improving the reproductive efficiency of the ewes. In addition, the embryo engineering technology can shorten the reproductive cycle of ewes and accelerate the breeding process, such that the breeding efficiency of a new sheep or goat breed can be improved by 50%-70%. Embryo engineering technology has become an essential support technology for building a breeding industry power.

Selection of oocyte donor animals is a key step in embryo engineering technology, as oocytes are the starting point for embryo development, and their quantity and quality directly influence the developmental potential of embryos and the success rate of embryo engineering process. In practice, the ovulation number of different donor ewes varies greatly after hormone induction, and some even do not ovulate, resulting in increased breeding costs, reduced economic benefits, and waste of human resources. Conventional methods for selecting oocyte donor animals depend on subjective evaluation, and the evaluation indicators mainly include information such as physical performance, the presence of diseases, age, previous litters, breed, lineage, etc., lacking a standardized procedure.

Therefore, the establishment of a more efficient, accurate, and standardized method for screening donor animals is an urgent problem to be solved in the field of livestock breeding.

### SUMMARY

To solve at least part of the technical problems in the prior art described above, the present disclosure provides a method for identifying a hyperovulatory donor ewe and use thereof.

In a first aspect of the present disclosure, provided is a method for identifying a hyperovulatory donor ewe, comprising the following steps:
(1) preparing a blood sample from a ewe;
(2) measuring γ-glutamyl transpeptidase activity, alkaline phosphatase activity, and/or platelet count in the blood sample to obtain respective measured values; and
(3) identifying the ewe as a hyperovulatory donor ewe based on the respective measured values.

In certain embodiments, the step (3) of the presently disclosed method comprises comparing each measured value with its corresponding predetermined threshold value.

In certain embodiments, wherein identifying the ewe as a hyperovulatory donor ewe when the measured value of γ-glutamyl transpeptidase activity is ≥its corresponding predetermined threshold, the measured value of alkaline phosphatase activity is ≥its corresponding predetermined threshold, and/or the measured value of platelet count is ≤its corresponding predetermined threshold.

In certain embodiments, the threshold value is a value statistically obtained from a sample group of age-matched donor ewes.

In certain embodiments, the threshold value is a value statistically obtained from a sample group of breed-matched ewes.

In certain embodiments, the ewe comprises a female sheep or a female goat.

In certain embodiments, the method further comprises separating serum from the blood sample before measuring activity of γ-glutamyl transpeptidase or alkaline phosphatase.

In certain embodiments, the blood sample is derived from venous blood of the ewe.

In a second aspect of the present disclosure, provided is use of the method according to the first aspect in large-scale propagation of genetically superior sheep breeds or in vitro embryo production.

In a third aspect of the present disclosure, provided is a kit for identifying a hyperovulatory donor ewe comprising a reagent for measuring γ-glutamyl transpeptidase activity, alkaline phosphatase activity, and/or platelet count in a blood sample from a ewe.

In a fourth aspect of the present disclosure, provided is an apparatus for identifying a hyperovulatory donor ewe comprising:
a data acquisition unit which being configured to acquire measured values of the γ-glutamyl transpeptidase activity, alkaline phosphatase activity, and/or platelet count in a blood sample from a ewe; and
a data processing unit which being configured to identify a hyperovulatory donor ewe based on the measured values.

In certain embodiments, predetermined threshold values are stored in the data processing unit for comparing the measured values with each corresponding predetermined threshold value.

In a fifth aspect of the present disclosure, provided is a computer-readable storage medium storing a computer program, wherein the computer program, when executed by a processor, implements:
acquiring measured values of γ-glutamyl transpeptidase activity, alkaline phosphatase activity, and/or platelet count in a blood sample from a ewe; and
identifying the ewe as a hyperovulatory donor ewe based on the measured values.

The present disclosure establishes a standardized method for identifying a hyperovulatory donor ewe by using γ-glutamyl transpeptidase activity, alkaline phosphatase activity, and/or platelet count as the evaluation indicators, thereby overcoming insufficient ovulation and large inter-individual variations among donor ewes caused by subjective judgment in conventional approaches. The method disclosed herein enables efficient screening of donor ewes, improves embryo production efficiency, reduces labor and material inputs, significantly lowers breeding costs, and enhances economic benefits. Accordingly, it is highly significant for promoting the advancement of livestock embryo production technology. Furthermore, the method features simple operation, high accuracy, high efficiency, and ease of popularization.

### BRIEF DESCRIPTION OF THE DRAWINGS

FIG. 1 illustrates the AUC evaluation results of γ-glutamyl transpeptidase activity, alkaline phosphatase activity, or platelet count as screening criteria for donor ewes.

FIG. 2 illustrates a technical roadmap of the present disclosure.

### DETAILED DESCRIPTION

Various exemplary embodiments of the present disclosure are described in detail below. The detailed description should not be construed as limitations on the present disclosure but as a more detailed description of certain aspects, features, and embodiments of the present disclosure.

It will be appreciated that the terms used in the present disclosure are for the purpose of illustrating embodiments only, rather than limiting the present disclosure. In addition, for the numerical ranges in the present disclosure, it will be appreciated that the upper and lower limits of the ranges are specifically disclosed, as well as every intervening value between them. Every smaller range between any stated value or intervening value in a stated range and any other stated or intervening value in that stated range is encompassed within the present disclosure. The upper and lower limits of these smaller ranges may independently be included or excluded in the range.

Unless defined otherwise, all technical and scientific terms used herein have the same meaning as commonly understood by those of ordinary skill in the art to which the present disclosure pertains. Although only preferred methods and materials are described herein, any methods and materials similar or equivalent to those described herein can be used in the practice or testing of the present disclosure. All documents described in this specification are incorporated by reference to disclose and describe the methods and/or materials associated with the in documents. In case of conflict with any incorporated document, the specification shall prevail.

As used in this specification and the appended claims, the singular forms “a”, “an”, and “the” include plural referents unless the context clearly dictates otherwise. Thus, for example, reference to “a component” includes one or more such components, and reference to “the component” refers to one or more such components previously identified.

### Method for Identifying a Hyperovulatory Donor Ewe

In one aspect of the present disclosure, provided is a method for identifying a hyperovulatory donor ewe comprising the following steps:
(1) preparing a blood sample from a ewe;
(2) measuring γ-glutamyl transpeptidase activity, alkaline phosphatase activity, and/or platelet count in the blood sample to obtain respective measured values; and
(3) identifying the ewe as a hyperovulatory donor ewe based on the respective measured values.

In one preferred embodiment, step (3) of the present disclosure comprises comparing the measured value with each corresponding predetermined threshold; for example, when the measured value of γ-glutamyl transpeptidase activity is ≥its predetermined threshold value, the measured value of alkaline phosphatase activity is ≥its predetermined threshold value, and/or the measured value of platelet count is ≤its predetermined threshold value, identifying the ewe as a hyperovulatory donor ewe.

In certain embodiments, the threshold value is statistically obtained from a sample group of age-matched donor ewes, or the threshold value is statistically obtained from a sample group of breed-matched ewes.

In one preferred embodiment, the threshold value of γ-glutamyl transpeptidase activity is 61.85 U/L, the threshold value of alkaline phosphatase activity is 73.85 U/L, and the threshold value of platelet count is 280×10⁹/L.

In a preferred embodiment, when the measured value of γ-glutamyl transpeptidase activity is ≥61.85 U/L, the measured value of alkaline phosphatase activity is ≥73.85 U/L, and/or the measured value of platelet count is ≤280×10⁹/L, the ewe is identified as a hyperovulatory donor ewe.

In the present disclosure, the serum γ-glutamyl transpeptidase activity, the serum alkaline phosphatase activity, and the blood platelet count may be separately used as individual evaluation indicator, or a combination of any two or three thereof may be used to identify a hyperovulatory donor ewe, which is not particularly limited.

In one specific embodiment, the method comprises the following steps:
(1) collecting a blood sample from a ewe, allowing the blood sample to stand, and performing centrifugation to obtain a serum;
(2) measuring γ-glutamyl transpeptidase activity in the serum to obtain a measured value; and
(3) identifying the ewe as la hyperovulatory donor ewe when the measured value of γ-glutamyl transpeptidase activity is ≥61.85 U/L.

In another specific embodiment, the method comprises the following steps:
(1) collecting a blood sample from a ewe, allowing the blood sample to stand, and performing centrifugation to obtain a serum;
(2) measuring alkaline phosphatase activity in the serum to obtain a measured value; and
(3) identifying the ewe as a hyperovulatory donor ewe when the measured value of the alkaline phosphatase activity is ≥73.85 U/L.

In yet another specific embodiment, the method comprises the following steps:
(1) collecting a blood sample from a ewe;
(2) detecting platelet count in the blood sample to obtain a measured value; and
(3) identifying the ewe as a hyperovulatory donor ewe when the measured value of platelet count is ≤280×10⁹/L.

In yet another specific embodiment, the method comprises the following steps:
(1) collecting a blood sample from a ewe, allowing the blood sample to stand, and performing centrifugation to obtain a serum;
(2) measuring γ-glutamyl transpeptidase activity and the serum alkaline phosphatase activity in the serum to obtain measured values; and
(3) identifying the ewe as a hyperovulatory donor ewe when the measured value of γ-glutamyl transpeptidase activity is ≥61.85 U/L and the measured value of alkaline phosphatase activity is ≥73.85 U/L.

In yet another specific embodiment, the method comprises the following steps:
(1) collecting a blood sample from a ewe, allowing the blood sample to stand, and performing centrifugation to obtain a serum;
(2) measuring γ-glutamyl transpeptidase activity and the blood platelet count in the serum to obtain measured values; and
(3) identifying the ewe as a hyperovulatory donor ewe when the measured value of γ-glutamyl transpeptidase activity is ≥61.85 U/L and the measured value of platelet count is ≤280× 10⁹/L.

In yet another specific embodiment, the method comprises the following steps:
(1) collecting a blood sample from a ewe, allowing the blood sample to stand, and performing centrifugation to obtain a serum;
(2) measuring alkaline phosphatase activity and the blood platelet count in the serum to obtain measured values; and
(3) identifying the ewe as a hyperovulatory donor ewe when the measured value of alkaline phosphatase activity is ≥73.85 U/L and the measured value of platelet count is ≤280×10⁹/L.

In yet another specific embodiment, the method comprises the following steps:
(1) collecting a blood sample from a ewe, allowing the blood sample to stand, and performing centrifugation to obtain a serum;
(2) measuring γ-glutamyl transpeptidase activity and alkaline phosphatase activity and blood platelet count in the serum to obtain measured values; and
(3) identifying the ewe as a hyperovulatory donor ewe when the measured value of γ-glutamyl transpeptidase activity is ≥61.85 U/L, the measured value of alkaline phosphatase activity is ≥73.85 U/L, and the measured value of platelet count is ≤280× 10⁹/L.

In the present disclosure, the breed of ewes is not particularly limited, and examples thereof comprises, but are not limited to a sheep, a goat, and the like.

During the standing or centrifugation in the foregoing steps, the standing time is 20-70 min, preferably 25-65 min, more preferably 30-60 min, e.g., 30, 35, 40, 45, 50, 55, or 60 min. The centrifugation speed is 2000-5000 r/min, preferably 2200-4800 r/min, more preferably 2400-4600 r/min, even more preferably 2600-4400 r/min, still more preferably 2800-4200 r/min, and most preferably 3000-4000 r/min, e.g., 3000, 3200, 3400, 3600, 3800, or 4000 r/min.

In certain embodiments, the method for detecting γ-glutamyl transpeptidase or alkaline phosphatase activity is not particularly limited, and methods known in the art can be used, of which the examples include, but are not limited to the colorimetric method, fluorescent probe method, enzyme-linked immunosorbent assay, and the like. The method for detecting platelet count is not particularly limited, and methods known in the art may be used, examples include but are not limited to microscopic counting, flow cytometry, impedance method, light scattering method, and the like.

### Use

In one aspect of the present disclosure, provided is use of the disclosed method in large-scale propagation of genetically superior sheep breeds or embryo production. The present disclosure screens and identifies donor ewes based on blood indicators for subsequent large-scale propagation of genetically superior breeds or embryo production. This enables rapid expansion of population of the elite ewe breed, substantial improvement in high-quality embryo production efficiency, and further enhancement of breeding economic benefits.

### Kit

In one aspect of the present disclosure, provided is a kit for identifying a hyperovulatory donor ewe comprising a reagent for detecting γ-glutamyl transpeptidase activity, alkaline phosphatase activity, and/or platelet count in a blood sample from a ewe.

In one specific embodiment, the kit further comprises at least one of an instruction manual, a standard substance, or a threshold card, wherein the instruction manual in the kit describes how to perform the identifying or screening steps and the criteria for identifying a hyperovulatory donor ewe.

The reagent in the disclosed kit is not particularly limited as long as it is capable of detecting γ-glutamyl transpeptidase activity, alkaline phosphatase activity, and/or platelet count. For example, the reagents for measuring γ-glutamyl transpeptidase activity include L-γ-glutamyl-3-carboxy-4-nitroaniline, glycylglycine, and magnesium ion. The reagents for measuring alkaline phosphatase activity include p-nitrophenyl phosphate, 2-amino-2-methyl-1-propanol (AMP) buffer, and magnesium ion. The reagents for measuring the platelet count include platelet-specific antibodies, such as fluorescently labeled anti-CD41, anti-CD61 antibodies and the like.

### Apparatus

In one aspect of the present disclosure, provided is an apparatus for identifying a hyperovulatory donor ewe, comprising:
a data acquisition unit which being configured to acquire a measured value of γ-glutamyl transpeptidase activity, alkaline phosphatase activity, and/or platelet count in a blood sample from a ewe; and
a data processing unit which being configured to identify a hyperovulatory donor ewe based on the measured value.

In certain embodiments of the disclosed apparatus, the data processing unit further comprises stored predetermined threshold values for comparing each of the measured values with its corresponding predetermined threshold value.

In certain embodiments, the disclosed apparatus further comprises a display unit (e.g., display screen) to display the screening results of the hyperovulatory donor ewes.

In one aspect of the present disclosure, provided is a computer-readable storage medium storing a computer program, wherein the computer program, when executed by a processor, implements:
acquiring measured value of γ-glutamyl transpeptidase activity, alkaline phosphatase activity, and/or platelet count in a blood sample from a ewe; and
identifying the ewe as a hyperovulatory donor ewe based on the measured values.

### Example 1

The establishment process of the present method for identifying a hyperovulatory donor ewe is described in detail below.

### 1. Materials

The methods used in the examples are conventional methods known to those skilled in the art unless otherwise specified. Reagents, consumables, and other materials used are all commercially available products unless otherwise specified.

### 2. Procedures

### 2.1 Selection of Ewe Breeds

A total of 400 ewes aged 2-4 years and weighing approximately 45-55 kg were selected, all individuals had clear lineage, good health status, good physical condition, regular estrous cycles, and were free of reproductive diseases.

### 2.2 Collection of Jugular Vein Blood Samples

The ewe's head was elevated and turned toward the side opposite the blood collection site by gently grasping its horns or ears. The wool at the sampling site was clipped, followed by disinfection of the exposed area. The mid-lower segment of the jugular groove was pressed with the left thumb to dilate the jugular vein. The blood vessel in the jugular groove was palpated with the right thumb or the index finger. The needle was rapidly penetrated through the skin into the blood vessel at an angle of 30-45° relative to the skin at the needle insertion site of the jugular groove. When a flashback was observed, the needle was inserted in a parallel direction and forward by 1-2 cm. 10 mL of blood was collected and the site of blood collection was disinfected with 5% iodine tincture.

### 2.3 Complete Blood Count (CBC)

CBC parameters, including white blood cell count (WBC), red blood cell count (RBC), hemoglobin (HGB), hematocrit (HCT), and platelet count (PLT), were determined using an automatic blood cell analyzer.

### 2.4 Preparation and Collection of Serum Samples

A 5 mL of blood sample was collected from the jugular vein of each donor ewe and allowed to stand at room temperature for 30-60 min. The sample was centrifuged at 3500 r/min for 10 min, after which the serum was isolated, aliquoted into 1.5 mL centrifuge tubes, and cryopreserved at −20 °C.

### 2.5 Determination of Serum Biochemical Parameters

Serum biochemical parameters, including γ-glutamyl transpeptidase (GGT), alkaline phosphatase (ALP), aspartate aminotransferase (AST), alanine aminotransferase (ALT), total protein (TP), albumin (ALB), globulin (GLB), creatinine (Cr), urea (Urea), potassium ion (K+), serum calcium (sCa), serum phosphorus (sP), serum magnesium (sMg), triglyceride (TG), total cholesterol (TC), high-density lipoprotein (HDL), low-density lipoprotein (LDL), glucose (Glu), β-hydroxybutyrate (β-HB), creatine kinase (CK), and lactate dehydrogenase (LDH), were determined using an automatic biochemical analyzer.

### 2.6 Determination of Serum Antioxidant Parameters

Serum antioxidant parameters, including total antioxidant capacity (T-AOC), glutathione peroxidase (GSH-Px) activity, malondialdehyde (MDA) content, and total superoxide dismutase (T-SOD) activity, were determined using a kit supplied by Nanjing Jiancheng Bioengineering Institute.

### 2.7 Measurement of Serum Hormone Levels

The levels of serum hormones, including follicle-stimulating hormone (FSH), estrogen (E2), and progestin (P4), were determined using an ELISA kit, and the OD value of each serum sample was measured at 450 nm on a microplate reader.

### 2.8 Follicle Hormone Induction in Donor Ewes

Progesterone implants were intravaginally implanted on any day in the estrus cycle of the ewes for 10 days. The hormone induction was started on day 10 and programmed as follows: FSH was injected once a day for 2 consecutive days at 240 IU per injection.

2.9 Ovum Pick-Up from Donor Ewes

Following the second injection of FSH, the donor ewes were deprived of food and water. After 48 hours, the donor ewes were anesthetized and fixed on a restraining support for laparoscopic ovum pick-up. Upon completion of ovum pick-up, progesterone implants were removed, and the number of retrieved oocyte of each donor ewe was recorded.

Donor ewes were divided into a hyperovulatory group (total number of oocyte>15) and a hypoovulatory group (total number of oocyte<10) based on the total number of retrieved oocyte.

### 2.10. Statistical Analysis

Experimental data were recorded in Microsoft Excel, and statistical software was employed for differential analysis.

### 3. Results

### 3.1 Analysis of CBC Parameters in Hyperovulatory and Hypoovulatory in Donor Ewes

As shown in Table 1, the platelet count in the hyperovulatory group was 182.53±8.16, which was significantly lower than that in the hypoovulatory group (363.11±11.47; P<0.01). No significant differences were observed in the remaining CBC parameters between the two groups (P>0.05).

TABLE 1
CBC parameters of donor ewes in two groups

| | Hyperovulatory | Hypoovulatory | |
|---|---|---|---|
| Index | group | group | P value |
| Platelet count (PLT, 10⁹/L) | 182.53 ± 8.16 | 363.11 ± 11.47 | 0.007** |
| Hematocrit (HCT, %) | 37.82 ± 1.07 | 38.41 ± 0.91 | 0.105 |
| Red blood cell count | 16.57 ± 0.52 | 15.72 ± 0.31 | 0.508 |
| (RBC, 10¹²/L) | | | |
| Hemoglobin (HGB, g/L) | 153.38 ± 8.07 | 161.00 ± 9.72 | 0.401 |
| White blood cell count | 11.39 ± 0.87 | 13.36 ± 2.02 | 0.638 |
| (WBC, 10⁹/L) | | | |

Note:
**indicates extremely significant difference (P < 0.01).

### 3.2 Analysis of Serum Antioxidant Parameters of Donor Ewes in Hyperovulatory and Hypoovulatory Groups

As shown in Table 2, no significant differences (P>0.05) were observed in all serum antioxidant parameters between the hyperovulatory and hypoovulatory groups.

TABLE 2
Serum antioxidant parameters of donor ewes in two groups

| | Hyperovulatory | Hypoovulatory | |
|---|---|---|---|
| Item | group | group | P value |
| Total antioxidant capacity | 0.62 ± 0.10 | 0.65 ± 0.15 | 0.605 |
| (T-AOC, U/mL) | | | |
| Total superoxide dismutase | 34.18 ± 3.62 | 31.60 ± 2.94 | 0.467 |
| (T-SOD, U/mL) | | | |
| Catalase (CAT, U/mL) | 4.23 ± 0.66 | 4.25 ± 5.63 | 0.523 |
| Glutathione peroxidase | 125.72 ± 9.73 | 118.91 ± 10.46 | 0.312 |
| (GSH-Px, U/mL) | | | |
| Malondialdehyde (MDA, | 6.17 ± 0.75 | 6.35 ± 0.92 | 0.253 |
| nmol/mL) | | | |

### 3.3 Analysis of Serum Biochemical Parameters of Donor Ewes in Hyperovulatory and Hypoovulatory Groups

As shown in Table 3, the serum activities of γ-glutamyl transpeptidase (GGT) and alkaline phosphatase (ALP) in the hyperovulatory group were 74.78=2.15 and 95.23±2.06, respectively, both values were extremely significantly higher than those in the hypoovulatory group (52.14±1.38 and 51.46±1.67; P<0.01). No significant differences were observed in the remaining serum biochemical parameters between the two groups.

TABLE 3
Serum biochemical parameters of donor ewes in two groups

| | Hyperovulatory | Hypoovulatory | |
|---|---|---|---|
| Index | group | group | P value |
| γ-Glutamyl transpeptidase (GGT, U/L) | 74.78 ± 2.15 | 52.14 ± 1.38 | 0.005** |
| Alkaline phosphatase (ALP, U/L) | 95.23 ± 2.06 | 51.46 ± 1.67 | 0.003** |
| Aspartate aminotransferase (AST, U/L) | 90.01 ± 2.67 | 81.43 ± 4.61 | 0.101 |
| Alanine aminotransferase (ALT, U/L) | 38.41 ± 4.43 | 39.13 ± 4.61 | 0.913 |
| Total protein (TP, g/L) | 72.32 ± 1.92 | 77.22 ± 2.12 | 0.105 |
| Albumin (ALB, g/L) | 25.50 ± 0.27 | 25.96 ± 0.99 | 0.648 |
| Globulin (GLB, g/L) | 46.76 ± 1.63 | 51.46 ± 1.51 | 0.071 |
| Creatinine (Cr, μmol/L) | 102.04 ± 4.21 | 93.73 ± 2.76 | 0.152 |
| Urea (mmol/L) | 3.00 ± 0.16 | 2.77 ± 0.12 | 0.294 |
| Potassium ion (K+, mmol/L) | 4.47 ± 0.10 | 4.28 ± 0.05 | 0.216 |
| Serum calcium (sCa, mmol/L) | 2.26 ± 0.04 | 2.30 ± 0.03 | 0.415 |
| Serum phosphorus (sP, mmol/L) | 2.42 ± 0.16 | 2.40 ± 0.13 | 0.937 |
| Serum magnesium (sMg, mmol/L) | 0.82 ± 0.03 | 0.81 ± 0.02 | 0.84 |
| Triglyceride (TG, mmol/L) | 0.28 ± 0.03 | 0.35 ± 0.03 | 0.158 |
| Total cholesterol (TC, mmol/L) | 1.96 ± 0.17 | 1.68 ± 0.14 | 0.255 |
| High-density lipoprotein (HDL, mmol/L) | 1.08 ± 0.11 | 0.91 ± 0.10 | 0.271 |
| Low-density lipoprotein (LDL, mmol/L) | 0.54 ± 0.04 | 0.51 ± 0.03 | 0.613 |
| Glucose (Glu, mmol/L) | 5.19 ± 0.91 | 5.22 ± 0.97 | 0.985 |
| β-hydroxybutyrate (β-HB, mmol/L) | 0.33 ± 0.03 | 0.30 ± 0.00 | 0.426 |
| Creatine kinase (CK, U/L) | 164.85 ± 51.67 | 128.36 ± 25.95 | 0.602 |
| Lactate dehydrogenase (LDH, U/L) | 426.33 ± 19.11 | 437.28 ± 16.17 | 0.686 |

Note:
**indicates extremely significant difference (P < 0.01).

### 3.4 Analysis of Serum Hormone Levels of Donor Ewes in Hyperovulatory and Hypoovulatory Groups

As shown in Table 4, no significant differences (P>0.05) in serum hormone levels were observed between the hyperovulatory and hypoovulatory groups.

TABLE 4
Analysis of serum hormone levels of donor ewes in two groups

| | Hyperovulatory | Hypoovulatory | |
|---|---|---|---|
| Indicators | group | group | P value |
| Follicle-stimulating hormone | 4.01 ± 0.53 | 5.52 ± 0.63 | 0.082 |
| (FSH, IU/mL) | | | |
| Estrogen (E₂, pg/mL) | 121.53 ± 23.04 | 115.12 ± 3.76 | 0.079 |
| Progestogen (P₄, ng/mL) | 9.54 ± 1.86 | 11.91 ± 3.39 | 0.523 |
| Creatine kinase (CK, U/L) | 164.85 ± 51.67 | 128.36 ± 25.95 | 0.602 |
| Lactate dehydrogenase | 426.33 ± 19.11 | 437.28 ± 16.17 | 0.686 |
| (LDH, U/L) | | | |

### 3.5 Serum γ-Glutamyl Transpeptidase and Alkaline Phosphatase Activity and Blood Platelet Count as Criteria for Screening Donor Ewes

Receiver operating characteristic (ROC) curve was used to evaluate the discriminatory ability of serum γ-glutamyl transpeptidase activity, alkaline phosphatase activity and the blood platelet count for screening donor ewes. The area under the curve (AUC) of the ROC curve was applied to characterize the capacity of these three indicators to distinguish hyperovulatory donor ewes.

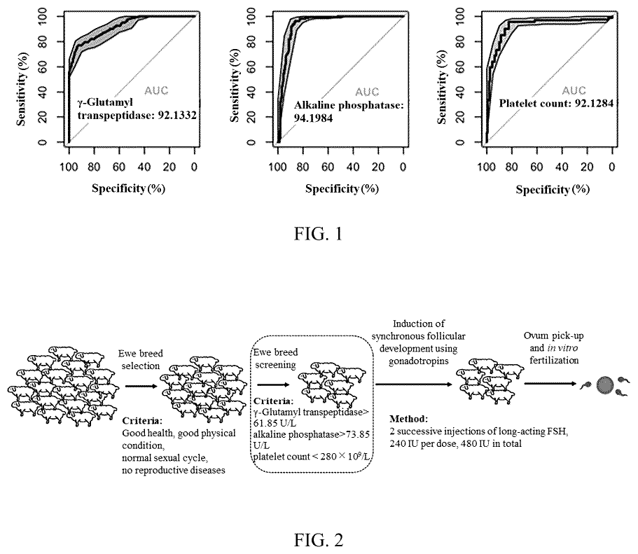

Generally, an AUC>0.7 indicates that the given indicator has a certain accuracy for the screening criterion results. In this example, 0.8 was used as a threshold value to improve the accuracy of the criterion. The ROC curve analysis revealed that the AUC of serum γ-glutamyl transpeptidase activity reached 0.92, the AUC of serum alkaline phosphatase activity reached 0.94, and the AUC of platelet count reached 0.92 (FIG. 1, Table 5). These results suggested that serum γ-glutamyl transpeptidase activity, alkaline phosphatase activity and platelet count can be used as the effective indicators for screening high-quality donor ewe. The detailed data are shown in Table 5.

TABLE 5
Thresholds of serum parameters for screening a donor ewe

| | γ-Glutamyl | Alkaline | Platelet count |
|---|---|---|---|
| Item | transpeptidase (GGT) | phosphatase (ALP) | (PLT) |
| Area under curve | 92.13 | 94.19 | 92.12 |
| (AUC, %) | | | |
| Threshold | >61.85 | >73.85 | <280 × 10⁹ |
| Sensitivity (%) | 75.78 | 96.27 | 95.65 |
| Specificity (%) | 93.75 | 86.25 | 82.5 |
| Youden's index | 0.701 | 0.813 | 0.824 |

When any threshold is selected, a trade-off is made between sensitivity and specificity.

As shown in Table 5, the sensitivity of γ-glutamyl transpeptidase activity was 75.78%, and the specificity was 93.75%, resulting in a Youden's index of 0.701; the sensitivity of alkaline phosphatase activity was 96.27%, and the specificity was 86.25%, resulting in a Youden's index of 0.813; and the sensitivity of platelet count was 95.65%, and the specificity was 82.5%, resulting in a Youden's index of 0.824. Such results suggest that when the serum γ-glutamyl transpeptidase activity of the donor ewe is greater than 61.85 U/L, the serum alkaline phosphatase activity is greater than 73.85 U/L, or the blood platelet count is less than 280×10⁹/L, the donor ewe can be selected for production use.

### 3.6 Validation of Parameters as Screening Criteria

To verify the accuracy of the serum γ-glutamyl transpeptidase and alkaline phosphatase activity and the blood platelet count as the screening criteria, 100 ewes were selected for predictive verification. The verification results, as shown in Table 6, indicate that the accuracies of the serum γ-glutamyl transpeptidase and alkaline phosphatase activity and the blood platelet count were 91.01%, 96.47%, and 93.41%, respectively. These results indicate that the proposed indicators exhibit excellent accuracy for screening donor ewes.

TABLE 6
Validation of indicators as screening criteria

| indicators | Number of caprines detected (n) | Accuracy (%) |
|---|---|---|
| γ-Glutamyl transpeptidase | 100 | 91.01 |
| Alkaline phosphatase | 100 | 96.47 |
| Platelet count | 100 | 93.41 |

In conclusion, through the analysis of a large number of experimental data, the present disclosure provides a method for screening donor ewes, which comprises detecting serum γ-glutamyl transpeptidase activity, serum alkaline phosphatase activity, and/or platelet count of a donor ewe, and enable to identify high-quality donor ewes according to corresponding threshold values.

### Example 2

This example illustrates a kit for identifying a hyperovulatory donor ewe, comprising at least a reagent for measuring γ-glutamyl transpeptidase activity, alkaline phosphatase activity, and/or platelet count in a blood sample from a ewe, together with a instruction manual for illustrating the implementation procedures of screening for the hyperovulatory donor ewe.

The kit may further comprise an additional reagent capable of performing a routine assay of various assay platforms, such as ELISA assays, immunoassays, protein chips or microarrays, DNA/RNA chips or microarrays, RT-PCR, the third generation of sequencing, mass spectrometry, immunohistochemistry, flow cytometry or cell screening.

In addition to the components described above, the kit of the present disclosure may further comprise precautions related to regulating the manufacture, use, or sale of a diagnostic kit in forms prescribed by governmental authorities. Additionally, the kit of the present disclosure may also be provided with detailed instructions for use, storage, and troubleshooting. The kit may optionally also be provided in a proper robotically operated apparatus, preferably for use in a high-throughput setting.

In certain embodiments, the components of the disclosed kit may be provided as a dry powder. When the agents and/or components are provided as a dry powder, the powder may be reconstituted by the addition of a proper solvent. It is contemplated that the solvent may also be disposed in another container. The container will typically comprise at least one vial, test tube, flask, bottle, syringe, and/or other container means in which the solvent is optionally aliquoted. The kit may further comprise a means for containing a second container of a sterile, pharmaceutically acceptable buffer and/or an additional solvent.

In certain embodiments, the components of the kit of the present disclosure may be provided in a solution, for example, an aqueous solution. In the case of an aqueous solution, the concentration or content of such components can be conveniently determined by those skilled in the art according to different requirements. For example, for storage purposes, for example, the component may be present in a form with a higher concentration, and when in operation or in use, the solution with the higher concentration may be, for example, diluted to reduce the concentration to a working concentration.

In the case where more than one component is present in the kit, the kit will generally further comprise second, third, or other additional containers into which the additional components can be separately placed. In addition, combinations of the components may be included in the containers.

### Example 3

This example illustrates an apparatus for identifying a hyperovulatory donor ewe, comprising at least:
a data acquisition unit which being configured to acquire measured values of γ-glutamyl transpeptidase activity, alkaline phosphatase activity, and/or platelet count in a blood sample from a ewe; and
a data processing unit which being configured to identify a hyperovulatory donor ewe based on the measured values,

In certain embodiments, the data processing unit further comprises predetermined threshold values for comparing each measured value with each corresponding predetermined threshold value.

It will be appreciated by those skilled in the art that various exemplary embodiments described herein may be implemented by software, or may be implemented by a combination of software and necessary hardware. Therefore, the specific embodiments according to the present disclosure may be embodied in the form of a software product that may be stored in a non-volatile storage medium or a non-transitory computer-readable storage medium (which may be a CD-ROM, a USB flash drive, a mobile hard disk, etc.) or on a network and comprise a number of instructions to cause a computing device (which may be a personal computer, a server, a mobile terminal, or a network device, etc.) to implement the method according to the present disclosure.

In an exemplary embodiment, the program product of the present disclosure may adopt any combination of one or more readable media. The readable medium may be a readable signal medium or a readable storage medium. The readable storage medium may be, for example, but is not limited to, an electrical, magnetic, optical, electromagnetic, infrared, or semiconductor system, apparatus, or device, or any combination thereof. More specific examples of the readable storage medium include, but are not limited to: an electrical connection having one or more wires, a portable disk, a hard disk, a random access memory (RAM), a read-only memory (ROM), an erasable programmable read-only memory (EPROM or a flash memory), an optical fiber, a portable compact disc read-only memory (CD-ROM), an optical storage device, a magnetic storage device, or any proper combination thereof.

The computer-readable storage medium in this example has a computer program stored thereon, which, when executed by a processor, implements:
acquiring measured values of γ-glutamyl transpeptidase activity, alkaline phosphatase activity, and/or platelet count in a blood sample from a ewe; and identifying the ewe as a hyperovulatory donor ewe based on the measured values.

Accordingly, based on the same inventive concept, the present disclosure further provides an electronic device.

In an exemplary embodiment, the electronic device is embodied in the form of a general-purpose computing device. Components of the electronic device may include, but are not limited to: at least one processor, at least one memory, and a bus connecting different system components (including the memory and the processor). The memory stores program codes executable by the processing unit to cause the processing unit to perform at least part of the steps of the method described herein. The processor comprises at least the data processing unit described herein. The memory may comprise a readable medium in the form of a volatile storage unit, e.g., a random access memory (RAM) and/or a cache storage unit, and may further comprise a read-only memory (ROM).

The memory of the present disclosure may further comprise a program/utility tool having a set of (at least one) program modules. Such program modules include, but are not limited to: an operating system, one or more application programs, other program modules, and program data, and each of these examples or certain combinations thereof may include the implementation of a network environment.

The bus may represent one or more of a plurality of bus structures, including a memory bus or memory controller, a peripheral bus, a graphics acceleration port, a processing unit, or a local bus using any bus structure in the plurality of bus structures.

The electronic device may also communicate with one or more external devices (for example, a keyboard, a pointing device, a Bluetooth device, etc.), and may also communicate with one or more devices that enable a user to interact with the electronic device, and/or communicate with any device (for example, a router, a modem, etc.) that enables the electronic device to communicate with one or more additional computing devices.

This communication may be performed through an input/output (I/O) interface. In addition, the electronic device may also communicate with one or more networks (such as a local area network (LAN), a wide area network (WAN), and/or a public network, such as the Internet) through a network adapter. The network adapter communicates with other modules of the electronic device through the bus. It will be appreciated that although not shown in this specification, other hardware and/or software modules may be used in combination with the electronic device, including, but not limited to, microcode, a device driver, a redundant processing unit, an external disk drive array, a RAID system, a tape drive, a data backup storage system, and the like.

Finally, it should be noted that the above examples are merely used for illustrating the technical solutions of the present disclosure, but not for limiting the present disclosure. Although the present disclosure is described in detail with reference to the aforementioned examples, those of ordinary skill in the art will appreciate that they may still make modifications to the technical solutions described in the aforementioned examples, or make equivalent replacements to some technical features in the examples. Nevertheless, such modifications or replacements do not render the essence of the corresponding technical solutions departing from the spirit and scope of the technical solutions of the examples of the present disclosure.

1-10. (canceled)

11. A method for identifying a hyperovulatory donor ewe comprising the following steps:
(1) preparing a blood sample from a ewe;
(2) measuring γ-glutamyl transpeptidase activity, alkaline phosphatase activity, and platelet count in the blood sample to obtain respective measured value; and
(3) identifying the ewe as a hyperovulatory donor ewe based on the respective measured value;
wherein the threshold value of γ-glutamyl transpeptidase activity is 61.85 U/L, the threshold value of alkaline phosphatase activity is 73.85 U/L, and the threshold value of platelet count is 280×10⁹/L, when the measured value of γ-glutamyl transpeptidase activity is ≥its corresponding predetermined threshold value, the measured value of alkaline phosphatase activity is ≥its corresponding predetermined threshold value, and the measured value of platelet count is ≤its corresponding predetermined threshold value, identifying the ewe as a hyperovulatory donor ewe.

12. The method according to claim 11, wherein the ewe comprises a female sheep or a female goat.

13. The method according to claim 11, wherein the method further comprises separating serum from the blood sample before measuring γ-glutamyl transpeptidase activity or alkaline phosphatase activity.

14. The method according to claim 11, wherein the blood sample is obtained by collecting venous blood of the ewe.

15. Use of the method according to claim 11 in large-scale propagation of genetically superior sheep breeds or embryo production.
